(19) United States
(12) Patent Application Publication
(10) Pub. No.: US 2009/0095622 A1
(43) Pub. Date: Apr. 16, 2009

(54) Biosensor and method to produce biosensor

(76) Inventors: Ching-Hsin Cho, Taipei Hsien (TW); Jui-Tsung Liao, Taipei Hsien (TW)

Correspondence Address: Andrews & Kurth, L.L.P., Suite 1100, 1350 I Street N.W., Washington, DC, 20005, US

(21) Appl. No.: 11/984,239

(22) Filed: Nov. 15, 2007

(30) Foreign Application Priority Data
Oct. 11, 2007 (TW) 096137937

(51) Int. Cl. G01N 33/487; G01N 33/49

(52) U.S. Cl. 204/403.01

(57) ABSTRACT

A biosensor is a sensor strip that is folded to form a base and a cover. The base has a reactive layer, two electrodes, a compensating electrode and a variable resistive pad. The reactive layer is formed on the base. The electrodes are formed on the base and connect to the reactive layer to an end of the base. The compensating electrode is formed between the electrodes and is connected to one electrode by the variable resistive pad having an adjustable resistivity to account for manufacturing errors. The cover is shorter than and adhered to the base and has a sample-dropping section corresponding to the reactive layer to allow a liquid sample to be added to the reactive layer. Operators insert the biosensor into the biosensor detector and obtain a correct result without calibrating the biosensor detector so preventing human calibration errors.

### BACKGROUND OF THE INVENTION

1. Field of Invention

The present invention relates to a biosensor and a method to produce the biosensor, and more particularly to a pre-calibrated biosensor.

2. Description of the Related Art

A patient who performs periodic blood tests (such as blood sugar measurement, triglyceride measurement, cholesterol measurement, high-density lipoprotein measurement, low-density lipoprotein measurement or the like.) has to go to hospital to have blood drawn before returning to hospital a few days later to obtain test results. Therefore, much of the patient's time is used traveling to hospital or testing, which is inconvenient. Moreover, the patient cannot obtain an immediate assessment of their physical condition, which can be dangerous for the patient. Biosensors and corresponding biosensor detectors having a display have been taught and allow a person to determine his or her physical condition virtually anywhere without trips to hospital. The biosensor is mounted in the biosensor detector and comprises a reactive layer. The reactive layer comprises an enzyme that reacts with a drop of the person's blood to generate an electric current. The biosensor detector detects the electric current and displays a test result on the display of the biosensor detector. Since the biosensor only requires a little blood and provides results quickly, the biosensor improves patient standard of living and safety.

A conventional biosensor comprises a sensor strip. The sensor strip is folded to form a base and a cover. The base has an inner surface, a proximal end, a distal end, a reactive layer, two electrodes and an identifying electrode. The reactive layer is mounted on the inner surface of the base and is coated with enzyme. The electrodes are mounted on the inner surface of the base and each electrode is formed from the reactive layer to the proximal end of the base. The identifying electrode is formed between the electrodes without contacting the reactive layer and allow the biosensor detector to recognize which test the biosensor performs (including, but not limited, to blood sugar measurement, triglyceride measurement, cholesterol measurement, high-density lipoprotein measurement, low-density lipoprotein measurement). The cover is shorter than the base, is bonded to the inner surface of the base, is flush with the distal end, covers the reactive layer of the base and leaves the proximal end of the base exposed. The cover has an inner surface and a through hole. The inner surface has a corresponding reactive layer. The corresponding reactive layer of the cover corresponds to the reactive layer of the base.

Because the reactive layer comprises an enzyme and different batches of enzyme that are produced at different times and have slightly different reactivity, the reactive layers with different batches of enzyme have relative errors in resistivity. Thus, the biosensor detector is required to be calibrated to correct the relative errors. Therefore, manufacturers need to mark a code on each biosensor to identify different batches of enzyme and produce a list comprising the codes and relative corrected data. Operators need to calibrate the biosensor detector by inputting the relative corrected data into the biosensor detector before inserting the biosensor. However, if operators forget to set the biosensor detector or input wrong relative corrected data, results of the biosensor detector are inaccurate. Moreover, it is inconvenient for operators and elongates a time of the process.

To overcome the shortcomings, the present invention provides a biosensor to mitigate or obviate the aforementioned.

### SUMMARY OF THE INVENTION

The primary objective of the present invention is to provide a pre-calibrated biosensor.

To achieve the objective, a biosensor in accordance with the present invention is folded to form a base and a cover. The base has a reactive layer, two electrodes, a compensating electrode and a variable resistive pad. The reactive layer is formed on the base. The electrodes are formed longitudinally on the base and connect to the reactive layer. The compensating electrode is formed between the electrodes. The variable resistive pad connects one electrode to the compensating electrode and has an adjustable resistivity to account for errors. The cover is shorter than and adhered to the base and has a sample-dropping section. The sample-dropping section corresponds to the reactive layer and allows blood or another liquid sample to be dropped on the reactive layer. Operators insert the biosensor into the biosensor detector and obtain a correct result without calibrating the biosensor detector therefore, preventing human calibration errors to improve patient standard of living and safety.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of a biosensor in accordance with the present invention with a first variant of a variable resistive pad;

FIG. 2 is a top view of the biosensor in FIG. 1, shown unfolded;

FIG. 3 is a top perspective view of a biosensor in accordance with the present invention with a sample-dropping section being a hole;

FIG. 4 is an enlarged top view of a biosensor in accordance with the present invention with a second variant of a variable resistive pad;

FIG. 5 is an enlarged top view of a biosensor in accordance with the present invention with a third variant of a variable resistive pad; and

FIG. 6 is an enlarged top view of a biosensor in accordance with the present invention with a fourth variant of a variable resistive pad.

### DETAILED DESCRIPTION OF THE INVENTION

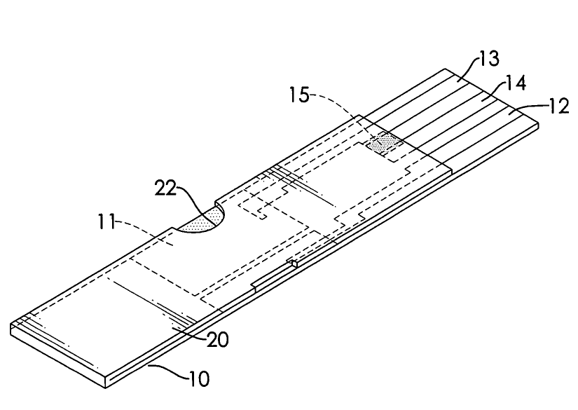

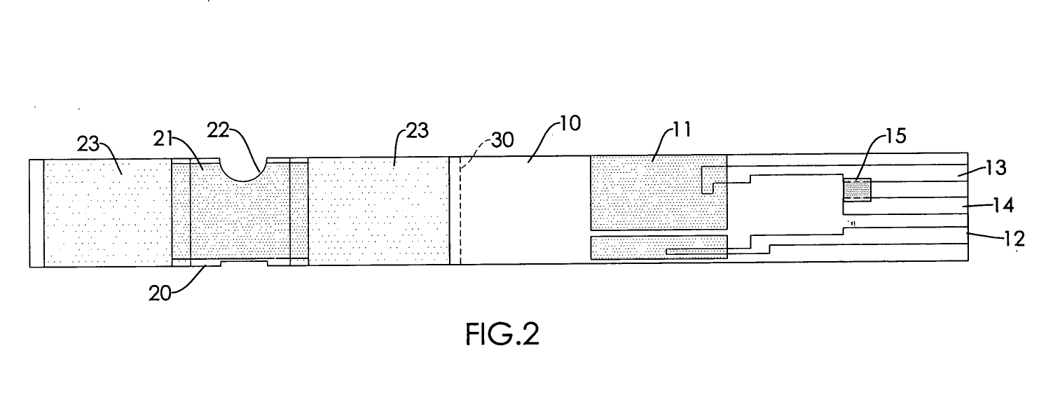

With reference to FIGS. 1 to 3, a biosensor in accordance with the present invention is a sensor strip. The sensor strip is folded along a folding line (30) and comprises a base (10) and a cover (20).

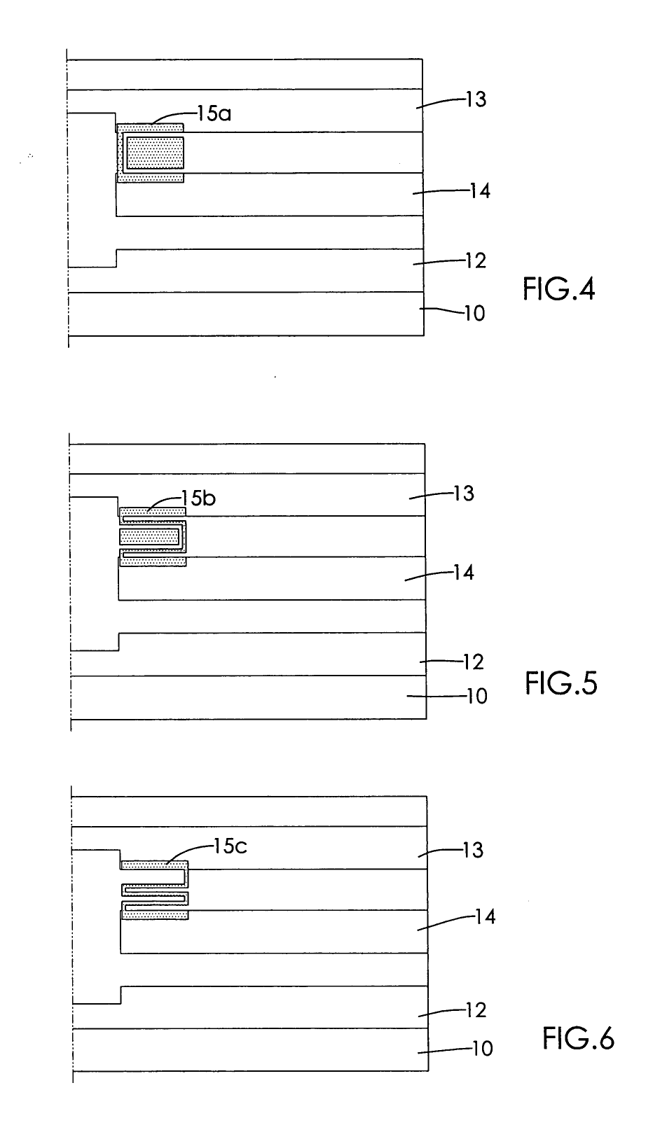

With further reference to FIG. 4 to 6, the base (10) has an inner surface, two sides, an end, a reactive layer (11), two electrodes (12, 13), a compensating electrode (14) and a variable resistive pad (15, 15a, 15b, 15c). The end of the base (10) is inserted into a biosensor detector.

The reactive layer (11) is formed on the inner surface of the base (10) and has an enzyme. For the biosensor to detect blood sugar, the reactive layer (11) may comprise a composition including: (A) an enzyme (such as glucose oxidase or the like); (B) enzyme protectors (such as albumin, dextrin, dextran, amino acid or the like); (C) a conductive medium (such as potassiumor the like); (D) a surfactant (such as triton X-100, triton X-405, triton X-114, sodium lauryl sulfate, tween 20 (polyoxyethylenesorbitan monolaurate), tween 40 (polyoxyethylenesorbitan monopalmitate), tween 60 (polyoxyethylenesorbitan monostearate), tween 80 (polyoxyethylenesorbitan monooleate), a water-soluble surfactant, a cleaning agent or the like); (E) a salt buffer (such as phosphateor the like); and (F) a solvent (such as distilled water).

The electrodes (12, 13) are formed on the inner surface, maybe respectively adjacent to the edges of the base (10) and in parallel, may be strips, are formed longitudinally on the reactive layer (11) and extend to the end of the base (10). The electrodes (12, 13) include an anode (12) and a cathode (13).). The cathode (13) is shorter than the anode (12). The compensating electrode (14) is formed substantially parallelly between the electrodes (12, 13) at the end of the base (10) without contacting the reactive layer (11).

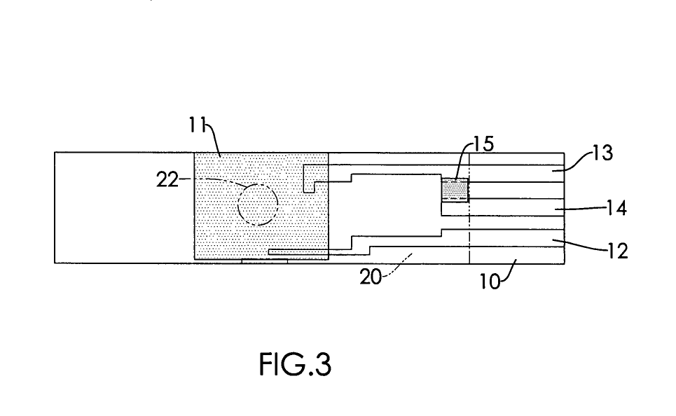

The variable resistive pad (15, 15a, 15b, 15c) connects the compensating electrode (14) to one electrode (12, 13), preferably connects the compensating electrode (14) to the cathode (13). Electric current passes through the variable resistive pad (15, 15a, 15b, 15c) from the cathode (13) to the compensating electrode (14). The variable resistive pad (15, 15a, 15b, 15c) has an area, a route and a resistivity. The route is defined as a flow of electricity, according to the principals of least resistance, through the variable resistive pad (15, 15a, 15b, 15c). The area and route are proportional to the resistivity and are calibrated to compensate for errors of manufacture. With reference to FIG. 3, a first variant of the carbon film resistance (15) is substantially rectangular.

With further reference to FIG. 4, a second variant of the carbon film resistance (15a) has a U-shaped route. The area of the carbon film resistance (15a) in the second variant is smaller than that of the carbon film resistance (15) in the first variant, so the resistivity of the carbon film resistance (15a) in the second variant is larger than that of the carbon film resistance (15) in the first variant.

With further reference to FIG. 5, a third variant of the carbon film resistance (15b) has an M-shaped route. On the premise that widths of the carbon film resistances (15a, 15b) in the second and the third variants are the same, a pathway of the carbon film resistance (15b) in the third variant is longer than that of the carbon film resistance (15a) in the second variant, so the resistivity of the carbon film resistance (15b) in the third variant is larger than that of the carbon film resistance (15a) in the second variant.

With further reference to FIG. 6, a fourth variant of the carbon film resistance (15b) has a four-bend route. On the premise that widths of the carbon film resistances (15b, 15c) in the third and the fourth variants are the same, a pathway of the carbon film resistance (15c) in the fourth variant is longer than that of the carbon film resistance (15b) in the third variant, so the resistivity of the carbon film resistance (15c) in the fourth variant is larger than that of the carbon film resistance (15b) in the third variant.

The cover (20) is shorter than the base (10), is bonded to the inner surface of the base (10), covers the reactive layer (11) and the variable resistive pad (15, 15a, 15b, 15c) of the base (10) and exposes the end of the base (10). The cover (20) has at least one edge, an inner surface, a corresponding reactive layer (21) and a sample-dropping section (22). The corresponding reactive layer (21) is formed on the inner surface of the cover (20) and multiple adhesive layers (23) 24 and corresponds to the reactive layer (11) of the base (10). The adhesive layers (23) are mounted on the inner surface of the cover (20), overlap partially the corresponding reactive layer (21) and bond to the inner surface of the base (10). The sample-dropping section (22) allows blood, urine, saliva or other such liquid or solution sample to be dropped on the reactive layer (11) and may be a hole or a notch. The hole is defined through the cover (20) and is formed centrally through the corresponding reactive layer (21). The notch is formed in one side of the cover (20) and corresponds to one electrode (12, 13) of the base (10), preferably the cathode (13).

A method to produce the biosensor in accordance with the present invention comprises steps of providing a sensor strip, printing a variable resistive pad, and folding the sensor strip.

The step of providing the sensor strip comprises obtaining the sensor strip with a reactive layer (11) of a base (10), a corresponding reactive layer (21) of a cover (20), two electrodes (12, 13), a compensating electrode (14) and a sample-dropping section (22). The sample-dropping section (22) corresponds to the reactive layer (11).

The step of printing the variable resistive pad comprises printing the variable resistive pad (15, 15a, 15b, 15c) on the biosensor strip to connect the compensating electrode (14) and one of the electrodes (12, 13). The variable resistive pad (15, 15a, 15b, 15c) may be substantially rectangular and may further be cut depending on a desired resistivity to form a U-shaped route, an M-shaped route, a multiple-bend route or any other shape by laser cutting technology. Altering a shape, area and route of the variable resistive pad (15, 15a, 15b, 15c) calibrates the resistivity to account for errors. The laser cutting technology is used to cut precision elements, such as resistors, capacitors or the like and is well known by those skilled in the art.

The step of folding the sensor strip comprises applying an adhesive and folding the sensor strip along a folding line (30) to allow the corresponding reactive layer (21) to overlap the reactive layer (11) and to expose outer ends of the electrodes (12, 13) and the compensating electrode (14) and hold the sensor strip together.

The biosensor comprises an applicable enzyme and the variable resistive pad (15, 15a, 15b, 15c) has a desired area, shape and route depending on errors of manufacture. Therefore, the variable resistive pad (15, 15a, 15b, 15c) has a desired resistivity to adjust relative errors of the reactive layer (11) and an operator inserts the biosensor directly into the biosensor detector to obtain a correct result without setting the biosensor detector every time before inserting the biosensor. Therefore, the biosensor is convenient, time saving and removes human calibration error from bio detector testing.

Even though numerous characteristics and advantages of the present invention have been set forth in the foregoing description, together with details of the structure and function of the invention, the disclosure is illustrative only. Changes may be made in detail, especially in matters of shape, size and arrangement of parts within the principles of the invention to the full extent indicated by the broad general meaning of the terms in which the appended claims are expressed.

What is claimed is:

1. A biosensor being a sensor strip being folded along a folding line and the biosensor and comprising
a base having
an inner surface;
an end;
a reactive layer being formed on the inner surface of the base;
two electrodes being formed on the inner surface of the base in parallel, on the reactive layer and extending to the end of the base;
a compensating electrode being formed substantially parallelly between the electrodes keeping free from contacting the reactive layer and at the end of the base; and
a variable resistive pad connecting the compensating electrode to one of the electrodes; and
a cover being shorter than the base, being bonded to the inner surface of the base, covering the reactive layer of the base, exposing the end of the base and having a sample-dropping section corresponding to the reactive layer of the base.

2. The biosensor as claimed in claim 1, wherein the variable resistive pad is substantially rectangular.

3. The biosensor as claimed in claim 1, wherein
the cover further has a corresponding reactive layer corresponding to the reactive layer of the base; and
the sample-dropping section is a notch being formed in one edge of the cover.

4. The biosensor as claimed in claim 2, wherein
the cover further has a corresponding reactive layer corresponding to the reactive layer of the base; and
the sample-dropping section is a notch being formed in one edge of the cover.

5. The biosensor as claimed in claim 1, wherein
the cover further has a corresponding reactive layer corresponding to the reactive layer of the base; and
the sample-dropping section is a hole that is defined through the cover and is formed centrally through the corresponding reactive layer.

6. The biosensor as claimed in claim 2, wherein
the cover further has a corresponding reactive layer corresponding to the reactive layer of the base; and
the sample-dropping section is a hole that is defined through the cover and is formed centrally through the corresponding reactive layer.

7. A method to produce a biosensor comprising:
obtaining a sensor strip having a reactive layer of a base, two electrodes, a compensating electrode and a sample-dropping section corresponding to the corresponding reactive layer;
printing the variable resistive pad on the biosensor strip to connect the compensating electrode and one of the electrodes; and
folding the sensor strip along a folding line to allow the corresponding reactive layer to overlap the reactive layer and to expose outer ends of the electrodes and the compensating electrode.

8. The method to produce a biosensor as claimed in claim 9, wherein the variable resistive pad is substantially rectangular.

9. The method to produce a biosensor as claimed in claim 10, wherein the variable resistive pad is cut depending on a desired resistivity by laser cutting technology before folding the sensor strip.

10. A biosensor comprising:
an anode;
a cathode;
an identifying electrode; and
a carbon film resistance connecting the identifying electrode with either the anode or the cathode and being treated to form at least one route to provide a specific resistivity.
